(19) United States
(12) Patent Application Publication
(10) Pub. No.: US 2024/0188839 A1
(43) Pub. Date: Jun. 13, 2024

(54) BLOOD PRESSURE ESTIMATION DEVICE AND CALIBRATION METHOD FOR BLOOD PRESSURE ESTIMATION DEVICE

(71) Applicant: OMRON HEALTHCARE Co., Ltd., Kyoto (JP)

(72) Inventors: Akito ITO, Kyoto (JP); Yasuhiro KAWABATA, Kyoto (JP); Kenji FUJII, Kyoto (JP); Naomi MATSUMURA, Kyoto (JP); Yuki SAKAGUCHI, Kyoto (JP)

(21) Appl. No.: 18/442,592

(22) Filed: Feb. 15, 2024

Related U.S. Application Data
(63) Continuation of application No. PCT/JP2022/041590, filed on Nov. 8, 2022.

(30) Foreign Application Priority Data
Nov. 10, 2021 (JP) 2021-182951

(51) Int. Cl. A61B 5/021; A61B 5/00; A61B 5/022; A61B 5/28

(52) U.S. Cl. CPC A61B 5/02125; A61B 5/02133; A61B 5/02208; A61B 5/28; A61B 5/7246; A61B 2560/0223; A61B 2562/0204

(57) ABSTRACT

Highly accurate blood pressure estimation based on a circulatory organ-related feature amount can be performed. A blood pressure estimation device includes a blood pressure estimation unit configured to acquire a circulatory organ-related feature amount that is a feature amount related to a state of a circulatory organ and changes in accordance with pulsation of a heart and to calculate a blood pressure value from the circulatory organ-related feature amount, and a reference blood pressure measurement unit including a sound wave detection unit configured to detect Korotkoff sound generated in accordance with the pulsation, the reference blood pressure measurement unit being configured to measure a reference blood pressure value by using the Korotkoff sound. The blood pressure estimation unit includes a feature amount acquisition unit configured to acquire the circulatory organ-related feature amount.

### CROSS-REFERENCE TO RELATED APPLICATIONS

This application is the U.S. national stage application filed pursuant to 35 U.S.C. 365(c) and 120 as a continuation of International Patent Application No. PCT/JP2022/041590, filed Nov. 8, 2022, which application claims priority to Japanese Patent Application No. 2021-182951, filed Nov. 10, 2021, which applications are incorporated herein by reference in their entireties.

### TECHNICAL FIELD

The present invention relates to a blood pressure estimation device configured to calculate blood pressure based on a circulatory organ-related feature amount, and a calibration method for the blood pressure estimation device.

### BACKGROUND ART

Conventionally, an upper arm or wrist sphygmomanometer using an oscillometric method has been used as means for calibrating a blood pressure estimation device based on a circulatory organ-related feature amount (see, for example, Patent Document 1).

### CITATION LIST

### Patent Literature

Patent Document 1: WO 2018/168790

### SUMMARY OF INVENTION

### Technical Problem

The oscillometric method is a method for calculating one blood pressure value from the entire time-series transition of a plurality of pulse pressure amplitude values measured during pressurization of a cuff. On the other hand, a plurality of circulatory organ-related feature amounts used for estimation of blood pressure are generally acquired during pressurization of the cuff. Therefore, when the circulatory organ-related feature amounts fluctuate during measurement of reference blood pressure values referred to at the time of calibration, the correspondence relationship between the circulatory organ-related feature amounts and the reference blood pressure values is not one-to-one, and calibration is not properly performed. Thus, the estimation accuracy of the blood pressure values may deteriorate.

In view of the problems of the related art as described above, an object of the present invention is to provide a blood pressure estimation device capable of performing highly accurate blood pressure estimation based on a circulatory organ-related feature amount, and a calibration method for the blood pressure estimation device.

### Solution to Problem

In order to solve the above problems, the present invention is a blood pressure estimation device that includes:
a blood pressure estimation unit configured to acquire a circulatory organ-related feature amount that is a feature amount related to a state of a circulatory organ and changes in accordance with pulsation of a heart and to calculate a blood pressure value from the circulatory organ-related feature amount; and
a reference blood pressure measurement unit including a sound wave detection unit configured to detect Korotkoff sound generated in accordance with the pulsation, the reference blood pressure measurement unit being configured to measure a reference blood pressure value by using the Korotkoff sound,
wherein the blood pressure estimation unit includes
a feature amount acquisition unit configured to acquire the circulatory organ-related feature amount,
a correspondence relationship determination unit configured to determine a correspondence relationship between the reference blood pressure value and an acquired value of the circulatory organ-related feature amount corresponding to a specific pulsation corresponding to the Korotkoff sound with which the reference blood pressure value in the pulsation is measured, and an estimated blood pressure acquisition unit configured to calculate the blood pressure value from the circulatory organ-related feature amount based on the correspondence relationship.

According to such a configuration, the blood pressure value is calculated from the circulatory organ-related feature amount based on the correspondence relationship between the reference blood pressure value measured by using the Korotkoff sound generated in accordance with the pulsation of the heart and the acquired value of the circulatory organ-related feature amount corresponding to the specific pulsation corresponding to the Korotkoff sound with which the reference blood pressure value in the pulsation is measured. Therefore, blood pressure can be calculated with high accuracy.

Further, in the present invention, the feature amount acquisition unit may include a pulse wave detection unit configured to detect a pulse wave.

According to such a configuration, various indexes that can be acquired based on the pulse wave can be used as the circulatory organ-related feature amount.

Furthermore, in the present invention the feature amount acquisition unit may include a first pulse wave detection unit and a second pulse wave detection unit configured to detect the pulse wave at two points having different pulse wave arrival times, and the feature amount acquisition unit may be configured to acquire, as the circulatory organ-related feature amount, a pulse transit time between the two points.

According to such a configuration, the blood pressure value can be estimated with high accuracy by using PTT (Pulse Transit Time) that indicates, as the circulatory organ-related feature amount, a pulse transit time between two points having different pulse wave arrival times.

Additionally, in the present invention the feature amount acquisition unit may include an electrocardiographic detection unit configured to detect an electrocardiogram and a vibration detection unit configured to detect vibration caused by the pulsation, and the feature amount acquisition unit may be configured to acquire, as the circulatory organ-related feature amount, a pulse transit time by using the pulse wave, the electrocardiogram, and the vibration.

According to such a configuration, the blood pressure value can be estimated with high accuracy by using the PTT that can be acquired based on the pulse wave, the electrocardiogram, and the vibration caused by the pulsation of the heart, as the circulatory organ-related feature amount. Here, the vibration caused by the pulsation of the heart includes sound waves and vibration generated on the body surface due to the pulsation of the heart, but is not limited thereto.

Further, in the present invention, the vibration detection unit may be the sound wave detection unit.

According to such a configuration, the blood pressure value can be estimated with high accuracy by using the PTT that can be acquired based on the sound wave, which is the vibration detected by the vibration detection unit and caused by the pulsation, the pulse wave, and the electrocardiogram.

Furthermore, in the present invention, the blood pressure estimation unit and the reference blood pressure measurement unit may be integrally configured.

According to such a configuration, an easy-to-handle biological state estimation device can be provided in which the blood pressure estimation unit and the reference blood pressure measurement unit are integrally configured.

Additionally, the present invention is a method for calibrating a blood pressure estimation device that is configured to calculate, based on a correspondence relationship between a circulatory organ-related feature amount that is related to a state of a circulatory organ and changes in accordance with pulsation of a heart and a blood pressure value, the blood pressure value from the circulatory organ-related feature amount, the method including:
detecting Korotkoff sound generated in accordance with the pulsation;
measuring a reference blood pressure value by using the Korotkoff sound;
acquiring the circulatory organ-related feature amount corresponding to a specific pulsation corresponding to the Korotkoff sound with which the reference blood pressure value in the pulsation is measured; and determining the correspondence relationship between the reference blood pressure value and the obtained circulatory organ-related feature amount.

According to such a configuration, the reference blood pressure value referred to at the time of calibrating the correspondence relationship used in the blood pressure estimation device configured to calculate, based on the correspondence relationship between the circulatory organ-related feature amount and the blood pressure value, the blood pressure value from the circulatory organ-related feature amount, is measured by using the Korotkoff sound generated in accordance with the pulsation of the heart, and the circulatory organ-related feature amount corresponding to the specific pulsation corresponding to the Korotkoff sound with which the reference blood pressure value is measured is calculated. The correspondence relationship between the reference blood pressure value and the calculated circulatory organ-related feature amount is determined as just described, making it possible to provide the calibration method capable of realizing highly accurate blood pressure estimation from the circulatory organ-related feature amount.

### Advantageous Effects of Invention

According to the present invention, highly accurate blood pressure estimation based on a circulatory organ-related feature amount can be performed.

### BRIEF DESCRIPTION OF DRAWINGS

FIG. 1 is a functional block diagram illustrating a blood pressure estimation device according to Example 1.

FIG. 2 is a flowchart illustrating the procedure of calibration processing according to Example 1.

FIG. 3 is a diagram illustrating the calibration processing according to Example 1.

FIG. 4 is a diagram illustrating an example of a correspondence relationship used for the calibration processing according to Example 1.

FIG. 5 is a flowchart illustrating the procedure of calibration processing according to Example 2.

FIG. 6 is a diagram illustrating the calibration processing according to Example 2.

FIG. 7 is a diagram illustrating an example of a correspondence relationship used for the calibration processing according to Example 2.

FIG. 8 is a functional block diagram illustrating a blood pressure estimation device according to Example 3.

FIG. 9 is a diagram illustrating an appearance configuration of the blood pressure estimation device according to Example 3.

### DESCRIPTION OF EMBODIMENTS

Embodiments of the present invention will be specifically described below with reference to the drawings.

### Example 1

Hereinafter, an example of the embodiments of the present invention will be described. It should be noted that the dimension, material, shape, relative arrangement, and the like of the components described in the present examples are not intended to limit the scope of this invention to them alone, unless otherwise stated.

### Configuration of Blood Pressure Estimation Device

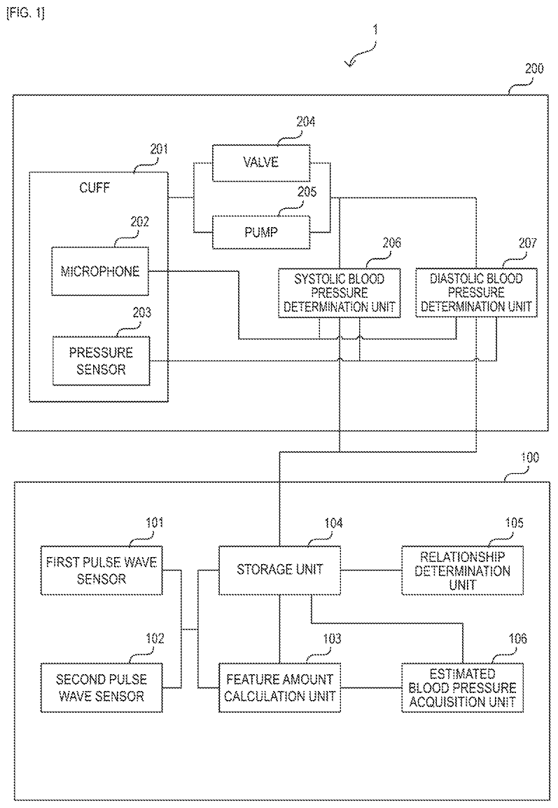

FIG. 1 is a functional block diagram illustrating a blood pressure estimation device 1 according to Example 1. The blood pressure estimation device 1 measures a pulse transit time (PTT) as a feature amount related to a circulatory organ (hereinafter referred to as a “circulatory organ-related feature amount”) in order to calculate blood pressure. PTT is the transit time of a pulse wave between two different points in an artery.

The blood pressure estimation device 1 includes a blood pressure estimation unit 100 and a reference blood pressure measurement unit 200. The blood pressure estimation unit 100 is a functional unit configured to acquire a PTT and calculate blood pressure from the acquired PTT. The reference blood pressure measurement unit 200 is a functional unit configured to measures, with high accuracy, blood pressure to be referred to at the time of calibrating a correspondence relationship between the PTT and the blood pressure, which will be described below.

### Blood Pressure Estimation Unit

The blood pressure estimation unit 100 includes a first pulse wave sensor 101, a second pulse wave sensor 102, a feature amount calculation unit 103, a storage unit 104, a relationship determination unit 105, and an estimated blood pressure acquisition unit 106.

The feature amount calculation unit 103, the storage unit 104, the relationship determination unit 105, and the estimated blood pressure acquisition unit 106 are actually configured to include a processor such as a CPU and a memory used as a workspace of the processor and a storage area of programs and data that are executed by the processor. The aforementioned functional units are realized through the execution of a predetermined program by the processor.

The first pulse wave sensor 101 and the second pulse wave sensor 102 are sensors configured to detect a pulse wave that is a waveform obtained by capturing changes in the pulse of the artery generated by pulsation of the heart. Here, the pulse wave includes a pressure pulse wave that is a waveform of internal pressure changes of the artery and a volume pulse wave that is a waveform of volume changes of the artery. A pulse wave sensor for detecting a pressure pulse wave includes a tonometry method, a piezoelectric method using a piezoelectric sensor, or the like. A pulse wave sensor for detecting a volume pulse wave includes an impedance method that detects a volume pulse wave as changes in impedance, a photoelectric method that detects volume changes by reflected light or transmitted light with the use of a light emitting element and a light receiving element, an electromagnetic irradiation method that detects volume changes as a phase shift between a transmitted wave and a reflected wave with the use of a transmitting element that transmits an electromagnetic wave and a receiving element that receives a reflected wave.

Sites of a subject on which the first pulse wave sensor 101 and the second pulse wave sensor 102 are placed can be set as appropriate; however, the first pulse wave sensor 101 is placed closer to the heart and the second pulse wave sensor 102 is placed farther away from the heart. In other words, the first pulse wave sensor 101 and the second pulse wave sensor 102 are placed at sites in which pulse wave arrival times to the same pulsation are different from each other, i.e., so as to be respectively placed upstream of the artery and downstream of the artery. Here, the first pulse wave sensor 101 and the second pulse wave sensor 102 correspond to a first pulse wave detection unit and a second pulse wave detection unit of the present invention, respectively, and both correspond to a pulse wave detection unit of the present invention.

The feature amount calculation unit 103 controls the first pulse wave sensor 101 and the second pulse wave sensor 102, and calculates the PTT by specifying the corresponding pulsation from the pulse waves detected by the first pulse wave sensor 101 and the second pulse wave sensor 102 (the pulse waves are referred to as a first pulse wave and a second pulse wave, respectively) with the use of a known method. Here, the first pulse wave sensor 101, the second pulse wave sensor 102, and the feature amount calculation unit 103 correspond to a feature amount acquisition unit of the present invention.

The storage unit 104 stores the first pulse wave and the second pulse wave in association with the time at which the pulse wave is detected. In addition, the storage unit 104 also acquires, from the reference blood pressure measurement unit 200, data such as Korotkoff sound, cuff pressure, systolic blood pressure, and diastolic blood pressure that are detected by the reference blood pressure measurement unit 200 described below, and stores the acquired data.

As will be described below, the relationship determination unit 105 determines a correspondence relationship between the PTT and the systolic blood pressure (SBP) based on the SBP measured by the reference blood pressure measurement unit 200 (the PTT corresponding to the SBP is referred to as PTTsbp). Here, the relationship determination unit 105 corresponds to a correspondence relationship determination unit of the present invention.

The estimated blood pressure acquisition unit 106 calculates the blood pressure from the PTT calculated by the feature amount calculation unit 103, based on the correspondence relationship between the PTT and the SBP acquired from the storage unit 104. Here, the estimated blood pressure acquisition unit 106 corresponds to a calibrated blood pressure acquisition unit of the present invention. In addition, the blood pressure estimation unit 100 corresponds to a blood pressure estimation unit of the present invention.

### Reference Blood Pressure Measurement Unit

The reference blood pressure measurement unit 200 includes a cuff 201, a microphone 202, a pressure sensor 203, a valve 204, a pump 205, a systolic blood pressure determination unit 206, and a diastolic blood pressure determination unit 207. Here, the reference blood pressure measurement unit 200 corresponds to a reference blood pressure measurement unit of the present invention. In addition, the microphone 202 corresponds to a sound wave detection unit of the present invention.

The systolic blood pressure determination unit 206 and the diastolic blood pressure determination unit 207 are actually configured to include a processor such as a CPU and a memory used as a workspace of the processor and a storage area of programs and data that are executed by the processor. The aforementioned functional units are realized through the execution of a predetermined program by the processor.

The reference blood pressure measurement unit 200 measures blood pressure by an auscultatory method. The auscultatory method is a method in which when the cuff 201 is depressurized from a state where the blood flow is stopped by pressurization by the cuff 201, Korotkoff sound generated by resumption of the blood flow is detected by the microphone 202, and blood pressure is measured based on the Korotkoff sound. By specifying the pulsation where the Korotkoff sound is generated, at what time the pulsation corresponding to the SBP has existed can be specified; therefore, a blood pressure value of even a blood pressure fluctuation in a short period of time such as a respiratory fluctuation can also be measured accurately at each time. An appropriate site such as the wrist or upper arm can be selected as a site of the subject on which the cuff 201 is placed.

The cuff 201 is a bag-shaped member inside of which air can be stored. By feeding air from the pump 205 into the cuff 201 in a state where the valve 204 is closed, the cuff 201 is pressurized. By opening the valve 204 from a state where the cuff 201 is pressurized, the air in the cuff 201 is discharged and the cuff 201 is thus depressurized. The microphone 202 for detecting the Korotkoff sound and the pressure sensor 203 for detecting the pressure in the cuff 201 are disposed in the cuff 201.

The systolic blood pressure determination unit 206 and the diastolic blood pressure determination unit 207 control the valve 204 and the pump 205, acquire the Korotkoff sound detected by the microphone 202 and the cuff pressure detected by the pressure sensor 203, and respectively determine systolic blood pressure SBP and diastolic blood pressure DBP by a known auscultatory method.

In the blood pressure estimation device 1, the blood pressure estimation unit 100 and the reference blood pressure measurement unit 200 may be integrally configured or may be separately configured. The blood pressure estimation unit 100 and the reference blood pressure measurement unit 200 are connected to each other by appropriate wired or wireless communication means. For example, the blood pressure estimation device 1 may be configured such that the blood pressure estimation unit 100 is a belt-shaped device to be wound around the upper arm, and such that the reference blood pressure measurement unit 200 is a wristwatch-type device to be wound around the wrist.

### Calibration Processing Procedure

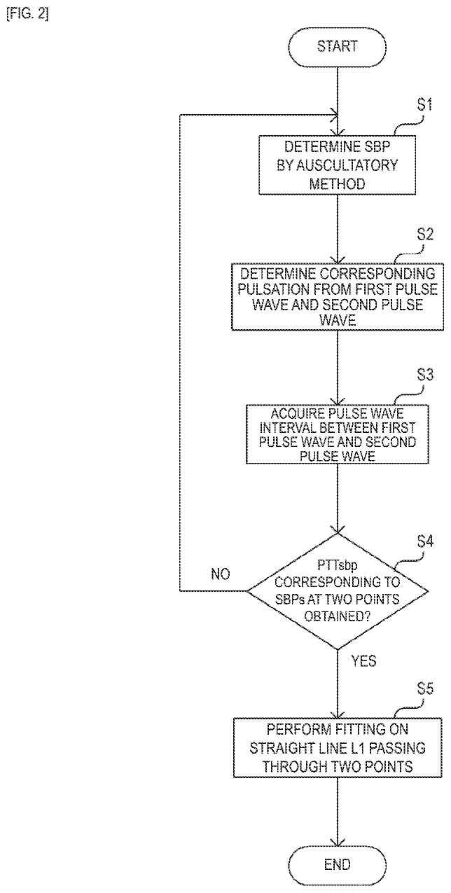

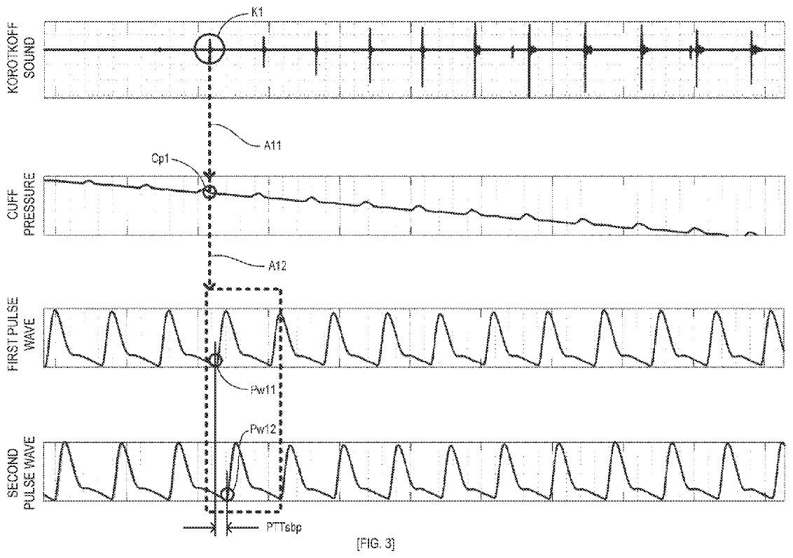

FIG. 2 is a flowchart illustrating the procedure for calibrating the blood pressure estimation device 1 according to Example 1. In addition, FIG. 3 is a graph showing the relationship among the Korotkoff sound, the cuff pressure, the first pulse wave, and the second pulse wave. In FIG. 3, the time courses of the Korotkoff sound and the cuff pressure on the horizontal axis are the same; however, these time courses and the time courses of the first pulse wave and the second pulse wave on the horizontal axis are not necessarily the same and indicate a relative time relation. The calibration processing illustrated in FIG. 2 corresponds to a calibration method of the present invention.

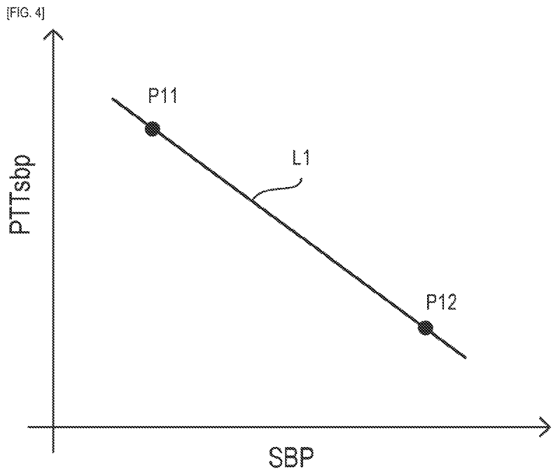

Note that in the present example, a linear relationship represented by a linear function as shown in FIG. 4 is assumed as the correspondence relationship between the PTT and the blood pressure. Pairs of PTTs and SBPs at two points are obtained, and fitting is performed on a straight line L1 connecting the two points to determine the correspondence relationship.

First, the systolic blood pressure determination unit 206 determines the SBP by the auscultatory method (step S1). More specifically, the pump 205 is operated to pressurize the cuff 201 to a predetermined pressure. The predetermined pressure is, for example, a value exceeding the systolic blood pressure by a predetermined value. The cuff 201 is pressurized to the predetermined pressure as just described, and thus the blood flow is stopped. From this state where the blood flow is stopped, the cuff 201 is gradually depressurized. When the cuff pressure decreases and the blood flow resumes, Korotkoff sound begins to be generated, and thus the first Korotkoff sound after the resumption of blood flow is detected by the microphone 202 (indicated by K1 in FIG. 3). Since cuff pressure Cp1 when the Korotkoff sound K1 is detected is the SBP, the SBP is determined based on the time when the Korotkoff sound K1 is detected, as indicated by dashed arrow A11 in FIG. 3. The SBP corresponds to a reference blood pressure value of the present invention.

Next, the relationship determination unit 105 determines a pulsation corresponding to the SBP from the first pulse wave and the second pulse wave that are stored in the storage unit 104, as indicated by dashed arrow A12 in FIG. 3 (step S2). Here, the pulsation corresponding to the SBP is a pulsation closest to the time when the Korotkoff sound K1 is detected.

Next, for the pulsation determined in step S2, the relationship determination unit 105 acquires a pulse wave interval PTTsbp between the first pulse wave and the second pulse wave, that is, the time interval between a first pulse wave Pw11 and a second pulse wave Pw12 (step S3). The pulse wave interval obtained as just described corresponds to the SBP and is therefore indicated by PTTsbp. Here, the PTTsbp corresponds to the acquired value of the circulatory organ-related feature amount of the present invention.

Next, the relationship determination unit 105 determines whether the PTTsbp corresponding to the SBPs at two points has been acquired (step S4). Also, when only the PTTsbp corresponding to one SBP has been acquired, the processing returns to step S1. When the PTTsbp corresponding to the SBPs at two points has been acquired, the processing goes to step S5.

Next, for the pairs of the SBPs at two points and the corresponding PTTsbp, the relationship determination unit 105 performs fitting on the straight line L1 passing through the two points (step S5). FIG. 4 is a graph illustrating an example of fitting with SBP on the horizontal axis and PTTsbp on the vertical axis. As shown in FIG. 4, a point obtained by plotting the SBP and the PTTsbp acquired by the processing of steps S1 to S3 in the first round is indicated by P11, and a point obtained by plotting the SBP and the PTTsbp acquired by the processing of steps S1 to S3 in the second round is indicated by P12. For example, as shown in FIG. 4, the correspondence relationship between the SBP and the PTTsbp can be represented with the straight line L1 passing through the two points P11 and P12.

By holding the correspondence relationship (straight line L1) between the SBP and the PTTsbp on which fitting is performed as just described in the storage unit 104, the estimated blood pressure acquisition unit 106 refers to the correspondence relationship and can continuously calculate the highly accurate SBP from the calculated values of the PTT continuously obtained by the feature amount calculation unit 103.

In addition, as described above, by obtaining the SBP corresponding to one specific pulsation by the auscultatory method, as a reference for calibrating the correspondence relationship between the PTT and the SBP, an accurate reference at each time can be obtained even when blood pressure fluctuation such as respiratory fluctuation occurs, and calibration processing can be performed in a short time.

The aforementioned calibration of the correspondence relationship between the PTT and the SBP may be performed, for example, every half hour to one hour; however, the timing of the calibration is not limited thereto. The correspondence relationship between the PTT and the SBP may be calibrated in response to an instruction from a user.

### Example 2

A blood pressure estimation device 2 according to Example 2 of the present invention will be described below. Configurations common to those in Example 1 are denoted by the same reference numerals, and detailed description thereof will be omitted.

The functional block diagram of the blood pressure estimation device 2 is the same as that of the blood pressure estimation device 1 illustrated in FIG. 1. In Example 1, the correspondence relationship between the PTT and the blood pressure is calibrated by using the systolic blood pressure SBP measured by the auscultatory method. However, in Example 2, the correspondence relationship between the PTT and the blood pressure is calibrated by using the diastolic blood pressure DBP measured by the auscultatory method.

### Calibration Processing Procedure

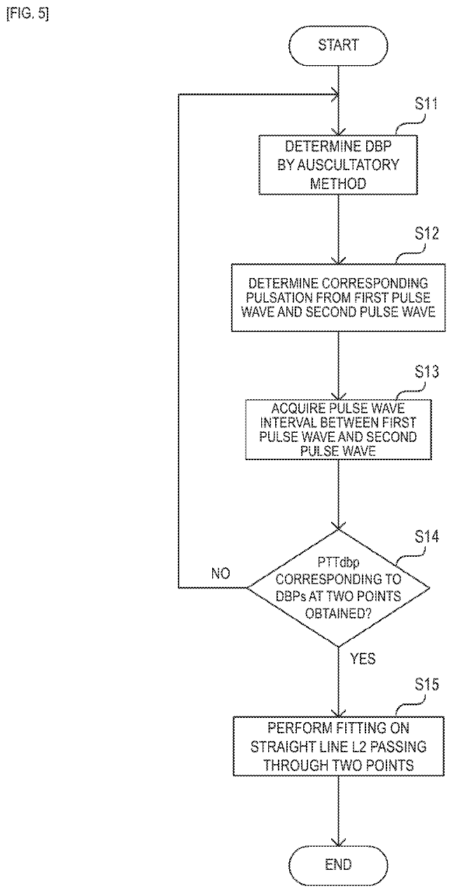

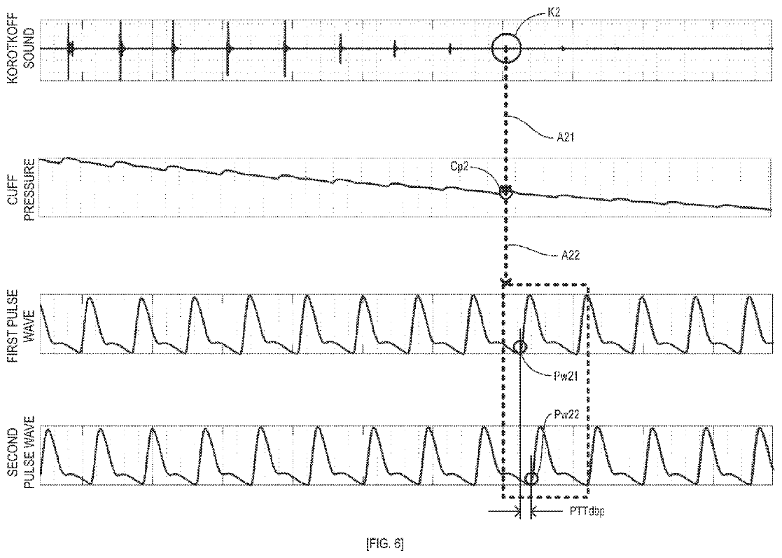

FIG. 5 is a flowchart illustrating the procedure for calibrating the blood pressure estimation device 2 according to Example 2. In addition, FIG. 6 is a graph showing the relationship among the Korotkoff sound, the cuff pressure, the first pulse wave, and the second pulse wave. In FIG. 6, the time courses of the Korotkoff sound and the cuff pressure on the horizontal axis are the same; however, these time courses and the time courses of the first pulse wave and the second pulse wave on the horizontal axis are not necessarily the same and indicate a relative time relation. The calibration processing illustrated in FIG. 5 corresponds to the calibration method of the present invention.

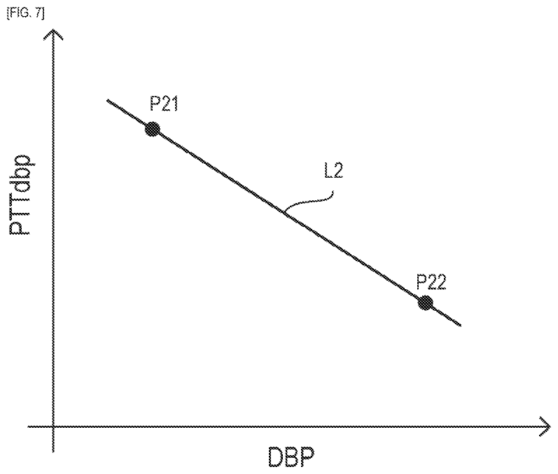

Note that in the present example, a linear relationship represented by a linear function as shown in FIG. 7 is assumed as the correspondence relationship between the PTT and the blood pressure. Pairs of PTT and DBPs at two points are obtained, and fitting is performed on a straight line L2 connecting the two points to determine the correspondence relationship.

First, the diastolic blood pressure determination unit 207 determines the DBP by the auscultatory method (step S11). More specifically, the cuff 201 is gradually depressurized from a predetermined pressure. The predetermined pressure can be appropriately set, and for example, can be set to a value lower than the systolic blood pressure by a predetermined value and to the pressure at which Korotkoff sound is generated. The cuff 201 is gradually depressurized from the predetermined pressure as just described, and thus the Korotkoff sound detected by the microphone 202 decreases. The cuff 201 is further depressurized, and then the Korotkoff sound disappears (indicated by K2 in FIG. 6). Since cuff pressure Cp2 when the Korotkoff sound K2 disappears is the DBP, the DBP is determined based on the time when the Korotkoff sound K2 disappears, as indicated by dashed arrow A21 in FIG. 6. The DBP corresponds to the reference blood pressure value of the present invention.

Next, the relationship determination unit 105 determines a pulsation corresponding to the DBP from the first pulse wave and the second pulse wave that are stored in the storage unit 104, as indicated by dashed arrow A22 in FIG. 6 (step S12). Here, the pulsation corresponding to the DBP is a pulsation closest to the time when the Korotkoff sound K2 disappeared.

Next, for the pulsation determined in step S12, a pulse wave interval PTTdbp between the first pulse wave and the second pulse wave, that is, the time interval between a first pulse wave Pw21 and a second pulse wave Pw22 is acquired (step S13). The pulse wave interval obtained as just described corresponds to the DBP and is therefore indicated by PTTdbp. Here, the PTTdbp corresponds to the acquired value of the circulatory organ-related feature amount of the present invention.

Next, the relationship determination unit 105 determines whether the PTTdbp corresponding to the DBPs at two points has been acquired (step S14). Also, when only the PTTdbp corresponding to one DBP is acquired, the processing returns to step S11. When the PTTdbp corresponding to the DBPs at two points is obtained, the processing goes to step S15.

Next, for the pairs of the DBPs at two points and the corresponding PTTdbp, the relationship determination unit 105 performs fitting on the straight line L2 passing through the two points (step S15). FIG. 7 is a graph illustrating an example of fitting with DBP on the horizontal axis and PTTdbp on the vertical axis. As shown in FIG. 7, a point obtained by plotting the DBP and the PTTdbp acquired by the processing of steps S11 to S13 in the first round is indicated by P21, and a point obtained by plotting the DBP and the PTTdbp acquired by the processing of steps S11 to S13 in the second round is indicated by P22. For example, as shown in FIG. 7, the correspondence relationship between the DBP and the PTTdbp can be represented with the straight line L2 passing through the two points P21 and P22.

By holding the correspondence relationship (straight line L2) between the DBP and the PTTdbp on which fitting is performed as just described in the storage unit 104, the estimated blood pressure acquisition unit 106 refers to the correspondence relationship and can continuously calculate the highly accurate DBP from the calculated values of the PTT continuously obtained by the feature amount calculation unit 103.

In addition, as described above, by obtaining the DBP corresponding to one specific pulsation by the auscultatory method, as a reference for calibrating the correspondence relationship between the PTT and the DBP, an accurate reference at each time can be obtained even when blood pressure fluctuation such as respiratory fluctuation occurs, and calibration processing can be performed in a short time.

The aforementioned calibration of the correspondence relationship between the PTT and the DBP may be performed, for example, every half hour to one hour; however, the timing of the calibration is not limited thereto. The correspondence relationship between the PTT and the DBP may be calibrated in response to an instruction from a user.

### Example 3

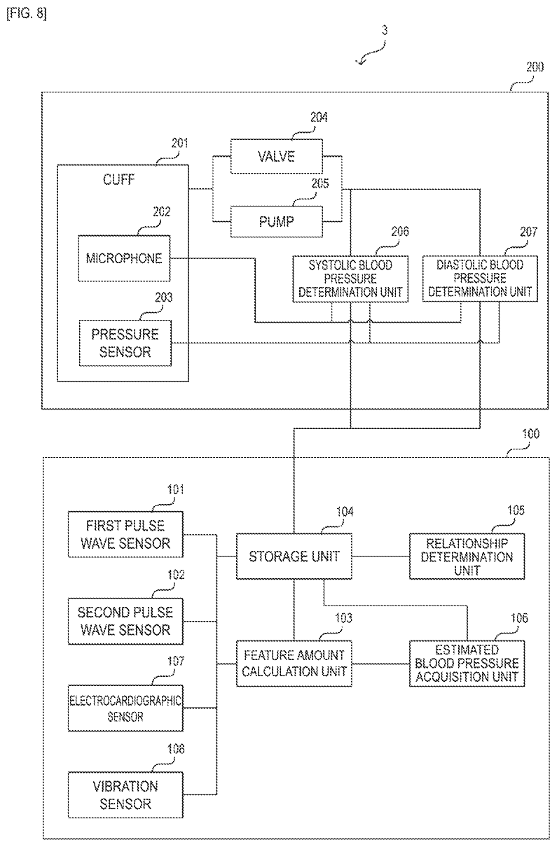

FIG. 8 illustrates a functional block diagram of a blood pressure estimation device 3 according to Example 3. Configurations common to those the blood pressure estimation device 1 according to Example 1 are denoted by the same reference numerals, and detailed description thereof will be omitted.

The blood pressure estimation device 3 is configured such that an electrocardiographic sensor 107 and a vibration sensor 108 are added to the blood pressure estimation device 1 according to Example 1. Although the blood pressure estimation device 3 illustrated in FIG. 8 includes the electrocardiographic sensor 107 and the vibration sensor 108, a configuration including at least one of the electrocardiographic sensor 107 and the vibration sensor 108 can be provided. In addition, here, the first pulse wave sensor 101 (and the second pulse wave sensor 102), the electrocardiographic sensor 107, the vibration sensor 108, and the feature amount calculation unit 103 correspond to the feature amount acquisition unit of the present invention.

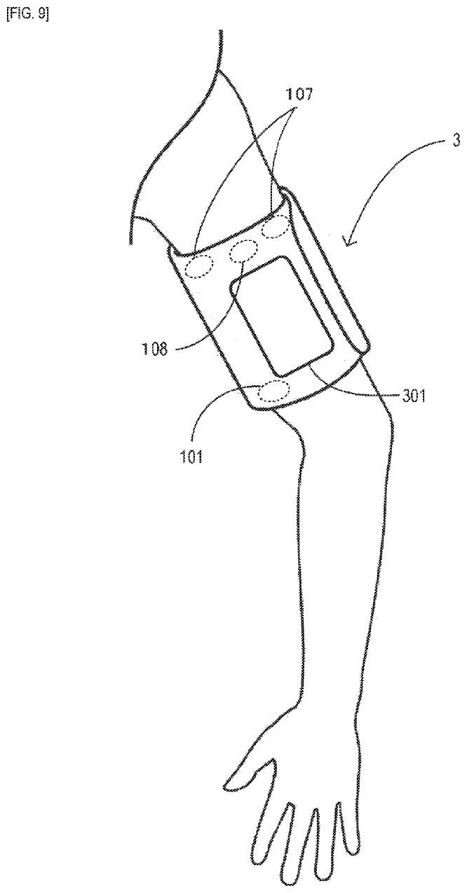

FIG. 9 illustrates a specific configuration example of the blood pressure estimation device 3. The blood pressure estimation device 3 has a belt shape to be wound around the upper arm, and the blood pressure estimation unit 100 and the reference blood pressure measurement unit 200 are integrally configured. The electrocardiographic sensors 107 are disposed on a subject side surface along an edge portion on the shoulder side of the blood pressure estimation device 3 wound along the upper arm, and the vibration sensor 108 is similarly disposed on the subject side surface along the edge portion on the shoulder side. The pulse wave sensor 101 (or the second pulse wave sensor 102) is disposed on the subject side surface along an edge portion on the elbow side of the blood pressure estimation device 3. The cuff 201 is disposed along the belt, and a mechanism unit such as the pump 205 and functional units such as the systolic blood pressure determination unit 206 and the feature amount calculation unit 103 are accommodated in a main body portion 301.

A pulse arrival time (PAT) can be measured by using the electrocardiographic sensors 107 and the pulse wave sensor 101. PAT is the pulse wave arrival time, and cardiac function can be evaluated by the PAT. The electrocardiographic sensor 107 corresponds to an electrocardiograhic detection unit of the present invention.

The PAT can be calculated as an interval between the time of an R wave of an electrocardiogram due to the pulsation of the heart detected by the electrocardiographic sensors 107 and the rising time of a pulse wave generated by the pulsation of the heart and detected by the pulse wave sensor.

The vibration sensor 108 is a sensor configured to detect vibration caused by the pulsation of the heart, that is, vibration generated on the body surface by transmission of the vibration caused by the pulsation of the heart. Specifically, the vibration sensor 108 can be configured by a microphone as a heart sound sensor configured to detect a sound wave that is vibration generated on the body surface by transmission of the vibration caused by the pulsation of the heart. In addition, as a ballistocardiogram sensor for detecting a ballistocardiogram that is vibration generated by pulsation of such vibration, the vibration sensor 108 can be specifically configured by an acceleration sensor, a piezoelectric sensor, or a strain gauge. The vibration detection method is not limited to such a detection method. A pulse-ejection period (PEP) can be measured by the vibration sensor 108. PEP is the time from the start of contraction of the left ventricle to the start of ejection into the aorta and is also referred to as pre-ejection time. Also, when a microphone is used as the vibration sensor 108, the microphone 202 can be substituted; therefore, the microphone 202 can be omitted by providing the vibration sensor 108. The vibration sensor 108 corresponds to a vibration detection unit of the present invention, and corresponds to a sound wave detection unit of the present invention when a microphone is used as the vibration sensor 108.

By providing the electrocardiographic sensors 107, the vibration sensor 108, and the first pulse wave sensor 101, the PAT and the PEP can be calculated as described above. In this case, since there is a relationship of PAT−PEP=PTT, the PTT can be calculated as the circulatory organ-related feature amount by the electrocardiographic sensors 107, the vibration sensor 108, and the first pulse wave sensor 101. In calculating the SBP or the DBP from the calculated values of the PTT, by obtaining the SBP or the DBP corresponding to one specific pulsation by the auscultatory method, as a reference for calibrating the correspondence relationship between the PTT and the SBP or the DBP in the same manner as in Example 1 or 2, an accurate reference at each time can be obtained even when blood pressure fluctuation such as respiratory fluctuation occurs, and calibration processing can be performed in a short time. In addition, since the PTT can be measured without providing two pulse wave sensors, one pulse wave sensor can be reduced and power saving can be achieved.

### Modified Example

In Example 1, Example 2, and Example 3, the PTT, the PAT, and the PEP are described as the circulatory organ-related feature amount; however, the circulatory organ-related feature amount is not limited thereto. For example, a pulse wave velocity (PWV), an augmentation index (Al), a left ventricular ejection time (LVET), blood pressure, a heart rate, or a heartbeat interval may be applied as the circulatory organ-related feature amount. Here, PPWV is the pulse wave velocity, Al is the pulse wave enhancement factor, and LVET is the left ventricular ejection time.

### REFERENCE NUMERALS LIST

1, 2, 3 Blood pressure estimation device
100 Blood pressure estimation unit
200 Reference blood pressure measurement unit
101 First pulse wave sensor
102 Second pulse wave sensor

1. A blood pressure estimation device comprising:
a blood pressure estimation unit configured to acquire a circulatory organ-related feature amount that is a feature amount related to a state of a circulatory organ and changes in accordance with pulsation of a heart and to calculate a blood pressure value from the circulatory organ-related feature amount; and
a reference blood pressure measurement unit including a sound wave detection unit configured to detect Korotkoff sound generated in accordance with the pulsation, the reference blood pressure measurement unit being configured to measure a reference blood pressure value by using the Korotkoff sound,
wherein the blood pressure estimation unit includes
a feature amount acquisition unit configured to acquire the circulatory organ-related feature amount,
a correspondence relationship determination unit configured to determine a correspondence relationship between the reference blood pressure value and an acquired value of the circulatory organ-related feature amount corresponding to a specific pulsation corresponding to the Korotkoff sound with which the reference blood pressure value in the pulsation is measured, and
an estimated blood pressure acquisition unit configured to calculate the blood pressure value from the circulatory organ-related feature amount based on the correspondence relationship.

2. The blood pressure estimation device according to claim 1, wherein the feature amount acquisition unit includes a pulse wave detection unit configured to detect a pulse wave.

3. The blood pressure estimation device according to claim 2, wherein the feature amount acquisition unit includes a first pulse wave detection unit and a second pulse wave detection unit configured to detect the pulse wave at two points having different pulse wave arrival times, the feature amount acquisition unit being configured to acquire, as the circulatory organ-related feature amount, a pulse transit time between the two points.

4. The blood pressure estimation device according to claim 2, wherein the feature amount acquisition unit includes an electrocardiogram unit configured to detect an electrocardiogram and a vibration detection unit configured to detect vibration caused by the pulsation, the feature amount acquisition unit being configured to acquire, as the circulatory organ-related feature amount, a pulse transit time by using the pulse wave, the electrocardiogram, and the vibration.

5. The blood pressure estimation device according to claim 4, wherein the vibration detection unit is the sound wave detection unit.

6. The blood pressure estimation device according to claim 1, wherein the blood pressure estimation unit and the reference blood pressure measurement unit are integrally configured.

7. A method for calibrating a blood pressure estimation device that is configured to calculate, based on a correspondence relationship between a circulatory organ-related feature amount that is related to a state of a circulatory organ and changes in accordance with pulsation of a heart and a blood pressure value, the blood pressure value from the circulatory organ-related feature amount, the method comprising:
detecting Korotkoff sound generated in accordance with the pulsation;
measuring a reference blood pressure value by using the Korotkoff sound;
acquiring the circulatory organ-related feature amount corresponding to a specific pulsation corresponding to the Korotkoff sound with which the reference blood pressure value in the pulsation is measured; and
determining the correspondence relationship between the reference blood pressure value and the obtained circulatory organ-related feature amount.
